(19) United States
(12) Patent Application Publication
(10) Pub. No.: US 2005/0107461 A1
(43) Pub. Date: May 19, 2005

(54) Drug combination therapy

(76) Inventors: Tian-Quan Cai, Edison, NJ (US); Yu-Sheng Chao, Warren, NJ (US)

Correspondence Address: MERCK AND CO., INC, P O BOX 2000, RAHWAY, NJ, 07065-0907, US

(21) Appl. No.: 10/507,048

(22) PCT Filed: Mar. 7, 2003
(86) PCT No.: PCT/US03/07038

Related U.S. Application Data
(60) Provisional application No. 60/363,442, filed on Mar. 12, 2002.

(51) Int. Cl. A61K 31/401; A61K 31/366; A61K 31/225

(52) U.S. Cl. 514/423; 514/460; 514/548

(57) ABSTRACT

The instant invention provides a drug combination comprised of an HMG-CoA reductase inhibitor in combination with an ACAT inhibitor, which is useful for treating or preventing Alzheimer's disease.

### FIELD OF THE INVENTION

The instant invention involves a drug combination comprising a 3-hydroxy-3-methylglutaryl coenzyme A (HMG-CoA) reductase inhibitor in combination with a compound which inhibits acyl-coenzyme A:cholesterol acyltransferase (ACAT) for the treatment and prevention of Alzheimer's disease.

### BACKGROUND OF THE INVENTION

Alzheimer's disease is a neurodegenerative disease of the brain leading to severely impaired cognition and functionality. This disease leads to progressive regression of memory and learned functions. Alzheimer's disease is a complex disease that affects cholinergic neurons, as well as serotonergic, noradrenergic and other central neurotransmitter systems. Manifestations of Alzheimer's disease extend beyond memory loss and include personality changes, neuromuscular changes, seizures, and occasionally psychotic features. Effective treatments for this devastating disease are not currently available.

One of the pathogenic events that occurs in identified inherited forms of Alzheimer's disease (AD) is the abnormal accumulation of amyloid β-peptide (Aβ). Aβ is produced during normal cellular processing of the Alzheimer amyloid precursor protein (APP) by β- and γ-secretase (Haass C, et al., Amyloid beta-peptide is produced by cultured cells during normal metabolism, Nature 359:322-5, 1992). Although Aβ40 is the major isoform of all the Aβ isoforms produced, about 10% of total Aβ consists of the two-amino acid longer form, Aβ42. Accumulating evidence indicates that an increase in the Aβ42/Aβ total ratio accelerates the aggregation and accumulation of Aβ into amyloid fibrils, leading to neurodegeneration and synaptic loss (Selkoe D J, Translating cell biology into therapeutic advances in Alzheimer's disease, Nature 399:A23-31, 1999; Tanzi E R, A genetic dichotomy model for the inheritance of Alzheimer's disease and common age-related disorders, J Clin Invest 104:1175-9, 1999). The molecular mechanisms that regulate APP processing and Aβ generation are still largely unknown.

HMG-CoA reductase is the rate-limiting enzyme for the synthesis of cholesterol. Inhibitors of HMG-CoA reductase, also known as stating, including marketed drugs such as lovastatin (MEVACOR®), simvastatin (ZOCOR®), pravastatin (PRAVACHOL®), fluvastatin (LESCOL®), and atorvastatin (LIPITOR®) lower plasma cholesterol and particularly low density lipoprotein (IDL) cholesterol.

Acyl-coenzyme A: cholesterol acyltransferase (ACAT) catalyzes the transfer of a fatty acid to the hydroxyl group of cholesterol that therefore becomes an ester. Since cholesterol esters are a key component of the atherosclerotic plaque and since cholesterol esterification may contribute to regulate cholesterol absorption, ACAT inhibitors have been sought as drug development candidates by a large number of pharmaceutical companies for the treatment of lipid disorders and atherosclerosis.

Epidemiological studies have suggested a relationship between serum cholesterol levels and AD. Recently, two independent reports showed a strong decrease in the incidence of AD for patients that were treated with inhibitors of HMG-CoA reductase (Wolozin B, et al., Decreased prevalence of Alzheimer disease associated with 3-hydroxy-3-methyglutaryl coenzyme A reductase inhibitors, Arch Neurol 57:143943, 2000; Jick H et al., Statins and the risk of dementia, Lancet 356:1627-31, 2000). Results from Fassbender et al. (Fassbender K et al., Simvastatin strongly reduces levels of Alzheimer's disease beta-amyloid peptides Abeta 42 and Abeta 40 in vitro and in vivo, Proc Natl Acad Sci USA. 98:5856-61, 2001) suggest that the beneficial effect from statins with respect to AD may derive from their inhibitory effect on the generation of AP. It was found that treatment with statins reduces Aβ42 in vitro and in vivo. More recently, studies from Puglielli et. al. (Puglielli L et al., Acyl-coenzyme A: cholesterol acyltransferase modulates the generation of the amyloid β-peptide, Nature Cell Biology 3:903-912,2001) showed that treatment with an ACAT inhibitor also reduces Aβ generation. Epidemiological studies have also indicated that inflammation can be a factor leading to the progression of Alzheimer's disease.

This invention provides a method for preventing or reducing Aβ formation, as well as reducing a patient's Aβ level. It also provides a pharmaceutical drug combination therapy comprised of an ACAT inhibitor in combination with an HMG-CoA reductase inhibitor for treating, preventing or reducing the risk for onset of Alzheimer's disease.

### SUMMARY OF THE INVENTION

This invention provides a pharmaceutical drug combination therapy comprised of a therapeutically or prophylactically effective amount of an HMG-CoA reductase inhibitor in combination with a therapeutically or prophylactically effective amount of an ACAT inhibitor.

The drug combination can be used in methods for preventing or reducing the risk for onset of Alzheimer's disease comprising administering a prophylactically effective amount of an HMG-CoA reductase inhibitor in combination with a prophylactically effective amount of an ACAT inhibitor to patient in need thereof.

The drug combination can also be used in methods for treating or slowing the progression of Alzheimer's disease comprising administering a therapeutically effective amount of an HMG-CoA reductase inhibitor in combination with a therapeutically effective amount of an ACAT inhibitor to patient in need thereof.

The drug combination can further be used in methods for preventing Aβ formation comprising administering a prophylactically effective amount of an HMG-CoA reductase inhibitor in combination with a prophylactically effective amount of an ACAT inhibitor to patient in need thereof.

The drug combination can also be used in methods for reducing Aβ formation, as well as for reducing a patient's Aβ level, comprising administering a therapeutically effective amount of an HMG-CoA reductase inhibitor in combination with a therapeutically effective amount of an ACAT inhibitor to patient in need thereof.

Additional objects of this invention will be evident from the following detailed description.

### DETAILED DESCRIPTION OF THE INVENTION

The present invention encompasses a method for treating, preventing or reducing the risk for onset of Alzheimer's disease in a patient comprising administering to said patient an HMG-COA reductase inhibitor in combination with a ACAT inhibitor in amounts that are effective to treat or prevent Alzheimer's disease.

A compound that inhibits HMG-CoA reductase is used in combination with an ACAT inhibitor to practice the instant invention. Compounds that have inhibitory activity for HMG-CoA reductase can be readily identified using assays well known in the art. For example, see the assays described or cited in U.S. Pat. No. 4,231,938 at col. 6, and WO 84/02131 at pp. 30-33, herein incorporated by reference.

Examples of HMG-CoA reductase inhibitors that may be used include but are not limited to lovastatin (MEVACOR®; see U.S. Pat. No. 4,231,938), simvastatin (ZOCOR®; see U.S. Pat. No. 4,444,784), pravastatin (PRAVACHOL®; see U.S. Pat. No. 4,346,227), fluvastatin (LESCOL®; see U.S. Pat. No. 5,354,772), atorvastatin (LIPITOR®; see U.S. Pat. No. 5,273,995), pitavastatin (also known as NK-104, see U.S. Pat. Nos. 5,284,953, 5,356,896 and 5,856,336), and rosuvastatin (also known as ZD-4522, see U.S. Pat. No. 5,260,440). The hemi-calcium salt of NK-104 is described and claimed in U.S. Pat. No. 5,856,336, and ZD-4522 is described in Drugs of the Future, 1999, 24(5), pp. 511-513, while the structural formulas of the other noted HMG-CoA reductase inhibitors, as well as additional examples of HMG-CoA reductase inhibitors, are described at page 87 of M. Yalpani, “Cholesterol Lowering Drugs”, Chemistry & Industry, pp. 85-89 (5 Feb. 1996). In general, HMG-CoA reductase inhibitors belong to a structural class of compounds which contain a moiety which can exist as either a 3-hydroxy lactone ring or as the corresponding 3,5-dihydroxy open-acid, and are commonly referred to as “statins.” The lactone portion of the statin and its corresponding dihydroxy open-acid form is shown below. 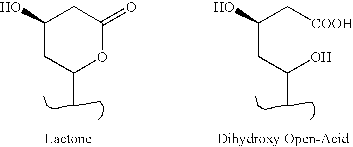
The term HMG-CoA reductase inhibitor is intended to include all lactone and open-ring 3,5-dihydroxy open-acid forms of HMG-CoA reductase inhibitors and the pharmaceutically acceptable salts and esters thereof; and therefor the use of such lactone and open-ring 3,5-dihydroxy acid forms and salts and esters thereof are included within the scope of this invention. Preferably, the HMG-CoA CoA reductase inhibitor is selected from lovastatin and simvastatin, which are lactonized statins, and their corresponding dihydroxy open acid forms and the pharmaceutically acceptable salts and esters thereof, and most preferably it is selected from simvastatin and its dihydroxy open acid form and the pharmaceutically acceptable salts and esters thereof, including for example the calcium and ammonium salts thereof.

Herein, the term “pharmaceutically acceptable salts” shall mean non-toxic salts of the compounds employed in this invention which are generally prepared by reacting the free acid with a suitable organic or inorganic base, particularly those formed from cations such as sodium, potassium, aluminum, calcium, lithium, magnesium, zinc and tetramethylammonium, as well as those salts formed from amines such as ammonia, ethylenediamine, N-methylglucamine, lysine, arginine, ornithine, choline, N,N′-dibenzylethylenediamine, chloroprocaine, diethanolamine, procaine, N-benzylphenethylamine, 1-p-chlorobenzyl-2-pyrrolidine-1′-yl-methylbenzimidazole, diethylamine, piperazine, morpholine, 2,4,4-trimethyl-2-pentamine and tris(hydroxymethyl)-aminomethane. Pharmaceutically acceptable esters at the carboxylic acid group can be made by treating a dihydroxy open acid statin with an alcohol. Examples of pharmaceutically acceptable esters of dihydroxy open acid statins include, but are not limited to, —C₁₋₄ alkyl and —C₁₋₄ alkyl substituted with phenyl-, dimethylamino-, and acetylamino. “C₁₋₄ alkyl” herein includes straight or branched aliphatic chains containing from 1 to 4 carbon atoms, for example methyl, ethyl, n-propyl, n-butyl, iso-propyl, sec-butyl and tert-butyl. Ester derivatives of the described compounds may act as prodrugs which, when absorbed into the bloodstream of a warm-blooded animal, may cleave in such a manner as to release the drug form and permit the drug to afford improved therapeutic efficacy.

A compound that inhibits ACAT is used in combination with an HMG-CoA reductase inhibitor to practice the instant invention. Recent work has demonstrated the existence of two distinct ACAT genes commonly referred to as ACAT-1 (sometimes referred to as ACAT I), described in U.S. Pat. No. 5,834,283, issued Nov. 10, 1998, and ACAT-2 (sometimes referred to as ACAT II), described in WO 97/45439 published Dec. 4, 1997. Compounds which have inhibitory activity for ACAT can be readily identified by using assays well-known in the art, for example as described in Chang C. C., Lee C. Y., Chang, E. T., Cruz, J. C., Levesque, M. C., Chang, T. Y.: J. Biol. Chem.;273: 35132-35141, 1998: Recombinant acyl-CoA:cholesterol acyltransferase-1 (ACAT-1) purified to essential homogeneity utilizes cholesterol in mixed micelles or in vesicles in a highly cooperative manner, herein incorporated by reference. As used herein, compounds which inhibit either or both of the isoforms of ACAT are included within the meaning of the term “ACAT inhibitor” (as well as within the meaning of similar phrases such as an “inhibitor of ACAT”, “a compound that inhibits ACAT” or the like) and are therefor within the scope of this invention. For example compounds which selectively or preferentially inhibit either ACAT-1 or ACAT-2, as well as compounds which have dual inhibitory activity for both ACAT-1 and ACAT-2, are useful with the present combination. Pharmaceutically acceptable salts and esters of ACAT inhibitors are likewise included within the scope of this invention.

Compounds which are ACAT inhibitors include but are not limited to those described in (i) U.S. Pat. No. 5,120,738 assigned to Fujirebio, Inc.; (ii) U.S. Pat. No. 5,340,807 assigned to Kyowa Hakkpo, Kogyo Co., Ltd.; (iii) U.S. Pat. No. 5,475,130 assigned to Taisho Pharmaceutical Co., Ltd.; (iv) U.S. Pat. No. 5,668,136 assigned to Eisai Co., Ltd.; (v) U.S. Pat. No. 5,760,087 assigned to Pierre Fabre Medicament; (vi) WO96/26925 applied for by Banyu Pharmaceutical Co., Ltd.; (vii) Sliskovic, D. R., CI-1011: An atypical ACAT inhibitor with antiatherosclerotic activity, Proceedings, XIVth International Symposium on Medicinal Chemistry, F. Awouters (editor) Elsevier Science B. V., 433-441, 1997 and WO97/16184 applied for by Warner-Lambert Co.; (viii) EP 0 635 501 A1 (European Application No. 94305305.8); and (ix) Tanaka, A. et al., Inhibition of acyl-CoA:cholesterol O-acyltransferase. 2. Identification and structure-activity relationship of a novel series of N-alkyl-N-(heteroaryl-substituted benzyl)-N′-arylureas, J. Med. Chem., 41:2390-2410, 1998, all of which are herein incorporated by reference.

Particular ACAT inhibitor compounds useful with this invention include Compounds (i)-(ix) shown below and the pharmaceutically acceptable salts and esters thereof: 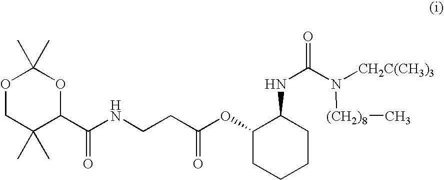
which is described in U.S. Pat. No. 5,120,738; 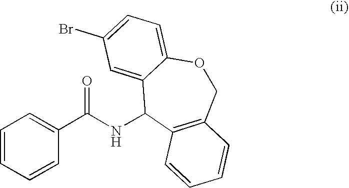
which is described in U.S. Pat. No. 5,340,807; 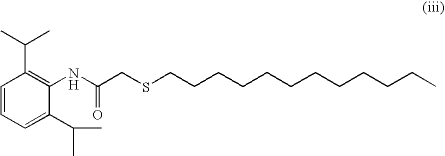
which is described in U.S. Pat. No. 5,475,130; 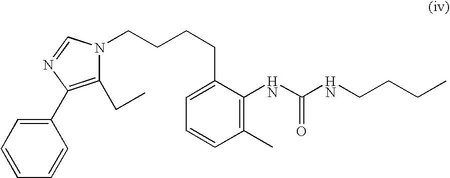
which is described in U.S. Pat. No. 5,668,136; 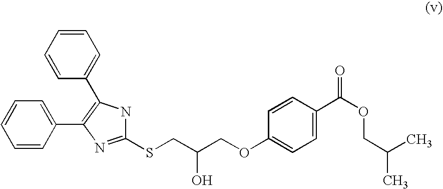
which is described in U.S. Pat. No. 5,760,087; 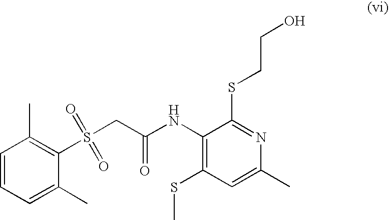
which is described in WO96/26925; 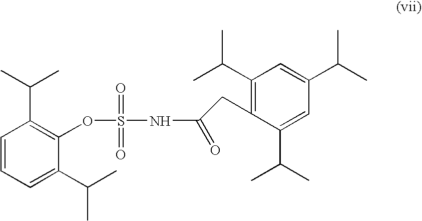
which is described in the D. R. Sliskovic publication noted above, 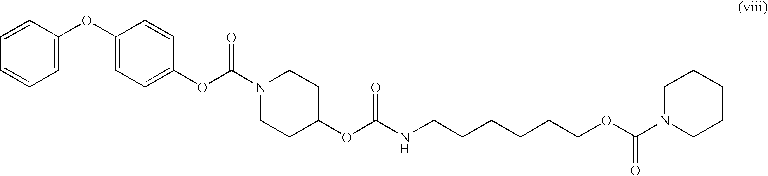
described in EP 0 635 501 A1 and 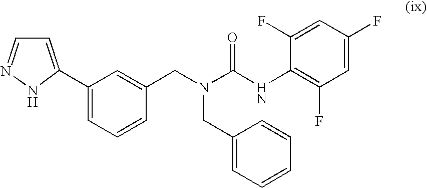
described in the A. Tanaka et al., publication noted above.

The compounds of use in this invention may have one or more chiral centers and the present compounds may occur as racemates, racemic mixtures and as individual diasteriomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

The ACAT inhibitors that may be used with this invention encompass all pharmaceutically acceptable salt forms of the compounds. Examples of such salt forms of ACAT inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The instant pharmaceutical combination comprising an HMG-CoA reductase inhibitor in combination with an ACAT inhibitor includes administration of a single pharmaceutical dosage formulation which contains both the HMG-CoA reductase inhibitor and the ACAT inhibitor, as well as administration of each active agent in its own separate pharmaceutical dosage formulation. Where separate dosage formulations are used, the HMG-CoA reductase inhibitor and the ACAT inhibitor can be administered at essentially the same time, i.e., concurrently, or at separately staggered times, i.e., sequentially. The instant pharmaceutical combination is understood to include all these regimens. Administration in these various ways are suitable for the present invention as long as the beneficial pharmaceutical effect of the HMG-CoA reductase inhibitor and the ACAT inhibitor are realized by the patient at substantially the same time. Such beneficial effect is preferably achieved when the target blood level concentrations of each active drug are maintained at substantially the same time. It is preferred that the HMG-CoA reductase inhibitor and the ACAT inhibitor be co-administered concurrently on a once-a-day dosing schedule; however, varying dosing schedules, such as the HMG-CoA reductase inhibitor once per day and the ACAT inhibitor once, twice or more times per day, is also encompassed herein. A single oral dosage formulation comprised of both an HMG-CoA reductase inhibitor and the ACAT inhibitor is preferred.

The term “patient” is intended herein to mean human patients who take an HMG-CoA reductase inhibitor in combination with an ACAT inhibitor for any of the uses described herein. Administering of the drug combination to the patient includes both self-administration and administration to the patient by another person.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician. The term “prophylactically effective amount” is intended to mean that amount of a pharmaceutical drug that will prevent or reduce the risk of occurrence of the biological or medical event that is sought to be prevented in a tissue, a system, animal or human by a researcher, veterinarian, medical doctor or other clinician. The present invention encompasses not only treating a patient who displays symptoms of Alzheimer's disease and reducing their level of Aβ, but also preventing the onset or progression of the disease and the formation of Aβ.

The dosage regimen utilizing an HMG-CoA reductase inhibitor in combination with an ACAT inhibitor is selected in accordance with a variety of factors including type, species, age, weight, sex and medical condition of the patient; the severity of the condition to be treated; the route of administration; the renal and hepatic function of the patient; and the particular compound or salt or ester thereof employed. Since two different active agents are being used together in a combination therapy, the potency of each of the agents and the interactive effects achieved by combining them together must also be taken into account. A consideration of these factors is well within the purview of the ordinarily skilled clinician for the purpose of determining the therapeutically effective or prophylactically effective dosage amounts needed to prevent, counter, or arrest the progress of the condition.

The daily dosage amounts of the HMG-CoA reductase inhibitor are intended to be the same or similar to those amounts which are employed for anti-hypercholesterolemic treatment and which are described in the Physicians' Desk Reference (PDR). For example, see the 56^{th} Ed. of the PDR, 2002 (Medical Economics Co.); in particular, see at page 208 the heading “Cardiovascular Agents,” sub-heading “Antilipemic Agents,” sub-sub-heading “HMG-CoA Reductase Inhibitors,” and the reference pages cited therein. For example, the oral dosage amount of HMG-CoA reductase inhibitor can be from about 0.1 to 200 mg/day, or from about 1 to 200 mg/day, preferably from about 0.1 to 100 mg/day, and more preferably from about 5 to 80 mg/day. However, dosage amounts will vary depending on the potency of the specific HMG-CoA reductase inhibitor used as well as other factors as noted above. An HMG-CoA reductase inhibitor which has sufficiently greater potency may be given in sub-milligram daily dosages. The HMG-CoA reductase inhibitor may be administered from 1 to 4 times per day, and preferably once per day.

As examples, the daily dosage amount for simvastatin may be selected from 5 mg, 10 mg, 20 mg, 40 mg and 80 mg; for lovastatin, 10 mg, 20 mg, 40 mg and 80 mg; for fluvastatin sodium, 20 mg, 40 mg and 80 mg; for pravastatin sodium, 10 mg, 20 mg, and 40 mg; and for atorvastatin calcium, 10 mg, 20 mg, and 40 mg.

The ACAT inhibitor can be administered to a patient in a dosage amount of 50 mg or less per day, for example from about 0.1 to 50 mg per day, particularly from 0.1 to 40 mg per day, more particularly from 0.1 to 30 mg per day, and most particularly from 0.1 to 20 mg per day. Examples of daily dosage amounts include but are not limited to 0.1, 1, 5, 10, 15, 20, 25, 30, 35, 40, 45 and 50 mg per day.

The instant combination therapy can be administered chronically in order to control the patient's Alzheimer's disease, and in order to gain the long-term benefits of Alzheimer's disease treatment and prevention. The drug combination can also be administered acutely when warranted.

Additional active agents may be used in combination with the HMG-CoA reductase inhibitor and ACAT inhibitor in a single dosage formulation, or may be administered to the patient in a separate dosage formulation, which allows for concurrent or sequential administration. One or more additional active agents may be administered with the instant combination therapy. The additional active agent or agents can be those useful for the treatment of neurodegenerative disorders and/or have other pharmaceutical activities. Examples of additional active agents which may be employed include HMG-CoA synthase inhibitors; squalene epoxidase inhibitors; squalene synthetase inhibitors (also known as squalene synthase inhibitors), probucol; niacin; fibrates such as clofibrate, fenofibrate, and gemfibrizol; a cholesterol absorption inhibitor such as ezetimibe which is 1-(4-fluorophenyl)-3(R)-[3(S)-(4-fluorophenyl)-3-hydroxypropyl)]-4(S)-(4-hydroxyphenyl)-2-azetidinone, described in U.S. Pat. Nos. 5,767,115 and 5,846,966; bile acid sequestrants; LDL (low density lipoprotein) receptor inducers; platelet aggregation inhibitors, for example glycoprotein IIb/IIIa fibrinogen receptor antagonists and aspirin; vitamin B₆ (also known as pyridoxine) and the pharmaceutically acceptable salts thereof such as the HCl salt; vitamin B₁₂ (also known as cyanocobalamin); beta-blockers; folic acid or a pharmaceutically acceptable salt or ester thereof such as the sodium salt and the methylglucamine salt; and anti-oxidant vitamins such as vitamin C and E and beta carotene.

Examples of HMG-CoA synthase inhibitors include: the beta-lactone derivatives disclosed in U.S. Pat. Nos. 4,806,564, 4,816,477, 4,847,271, and 4,751,237; the beta lactam derivatives disclosed in U.S. Pat. No. 4,983,597 and the substituted oxacyclopropane analogues disclosed in European Patent Publication EP 0 411 703. The squalene synthetase inhibitors suitable for use herein include, but are not limited to, those disclosed by Biller et al., J. Med. Chem., 1988 Vol. 31, No. 10, pp. 1869-1871, including isoprenoid (phosphinylmethyl)-phosphonates such as those of the formula 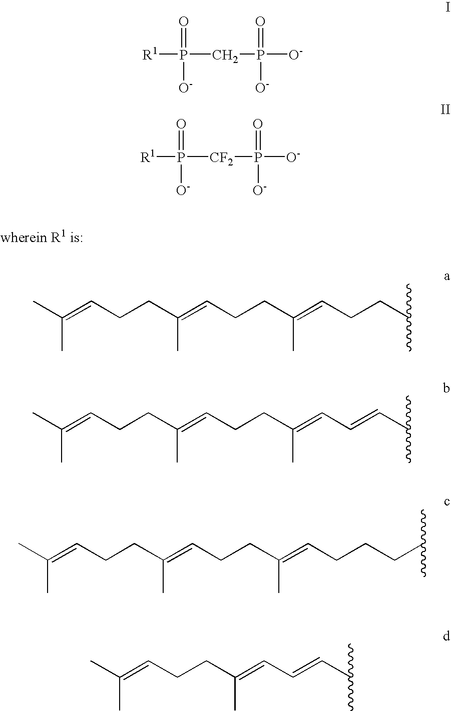
including the triacids thereof, triesters thereof and tripotassium and trisodium salts thereof as well as other squalene synthetase inhibitors disclosed in pending U.S. Pat. Nos. 4,871,721 and 4,924,024 and in Biller et al., J. Med. Chem., 1988, Vol. 31, No. 10, pp. 1869 to 1871.

In addition, other squalene synthetase inhibitors suitable for use herein include the terpenoid pyrophosphates disclosed by P. Ortiz de Montellano et al., J. Med. Chem., 1977, 20, 243-249, the farnesyl diphosphate analog A and presqualene pyrophosphate (PSQ-PP) analogs as disclosed by Corey and Volante, J. Am. Chem. Soc. 1976, 98, 1291-1293, phosphinylphosphonate reported by McClard, R. W. et al., J.A.C.S., 1987, 109, 5544 and cyclopropanes reported by Capson, T. L., PhD dissertation, June, 1987, Dept. Med. Chem. U. of Utah, Abstract, Table of Contents, pp. 16, 17, 4043, 48-51, Summary.

Further, the benzodiazepine squalene synthase inhibitors described in EP 0 567 026 to Takeda Chemical Industries, and the quinuclidinyl squalene synthase inhibitors described in PCT publications WO 94/03451, WO 93/09115, WO 93/21183, WO 93/21184, WO 93/24486, and U.S. Pat. No. 5,135,935, may be co-administered with the HMG-CoA reductase inhibitor plus ACAT inhibitor combination of the present invention. In addition, the zaragozic acid type squalene synthase inhibitors as described in U.S. Pat. Nos. 5,284,758; 5,283,256; 5,262,435; 5,260,332; 5,264,593; 5,260,215; 5,258,401; 5,254,727; 5,256,689; 5,132,320; 5,278,067, and PCT Publications WO 92/12156; WO 92/12157; WO 92/12158; WO 92/12159; WO 92/12160; WO 93/18040; WO 93/18039; WO 93/07151; and European Patent Publications EP 0 512 865, EP 0 568 946; EP 0 524,677 and EP 0 450 812, as well as the acyclic tricarboxylic acid compounds of U.S. Pat. No. 5,254,727, may be employed.

Illustrative examples of squalene epoxidase inhibitors are disclosed in European Patent Publication EP 0 318 860 and in Japanese Patent Publication JO2 169-571A. LDL-receptor gene inducer molecules are disclosed in U.S. Pat. No. 5,182,298.

Examples of bile acid sequestrants which may be employed in the present method include cholestyramine, colestipol, and poly [methyl-(3-trimethylaminopropyl)imino-trimethylene dihalide] and those disclosed in WO95/34585 to Geltex Pharmaceuticals, Inc. and EP 0 622 078 assigned to Hisamitsu Pharmaceutical Co., Inc.

Examples of cholesterol absorption inhibitors which may be employed in the present method include those described in WO 95/18143 and WO 95/18144 both assigned to Pfizer Inc., and WO 94/17038, WO 95/08532 and WO 93/02048 each assigned to Schering Corp.

The additional active agents described above which may be employed along with the HMG-CoA reductase inhibitor and ACAT inhibitor combination therapy can be used, for example, in amounts as indicated in the PDR or in amounts as indicated in the reference disclosures, as appropriate.

The active agents employed in the instant combination therapy can be administered in such oral forms as tablets, capsules, pills, powders, granules, elixirs, tinctures, suspensions, syrups, and emulsions. The instant invention includes the use of both oral rapid-release and time-controlled release pharmaceutical formulations, as well as enteric coated formulations. A particular example of an oral time-controlled release pharmaceutical formulation is described in U.S. Pat. No. 5,366,738. Oral formulations are preferred. Such pharmaceutical compositions are known to those of ordinary skill in the pharmaceutical arts; for example, see Remington's Pharmaceutical Sciences, Mack Publishing Co., Easton, Pa.

In the methods of the present invention, the active agents are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

Although the active agents of the present method may be administered in divided doses, for example two or three times daily, a single daily dose of each of the HMG-CoA reductase inhibitor and the ACAT inhibitor is preferred, with a single daily dose of both agents in a single pharmaceutical composition being most preferred.

The instant invention also encompasses a process for preparing a pharmaceutical composition comprising combining the BMG-CoA reductase inhibitor and the ACAT inhibitor with a pharmaceutically acceptable carrier, as well as the pharmaceutical composition which is made by combining the HMG-CoA reductase inhibitor and the ACAT inhibitor with a pharmaceutically acceptable carrier.

Therapeutically effective amounts of antihypertensive compound or compounds and cholesterol absorption inhibitor can be used together for the preparation of a medicament useful for treating or preventing any of the medical conditions described herein, in dosage amounts described herein. For example, the medicament may be useful for treating hypertension, preventing or reducing the risk of developing atherosclerotic disease, halting or slowing the progression of atherosclerotic disease once it has become clinically manifest, and preventing or reducing the risk of a first or subsequent occurrence of an atherosclerotic disease event.

The instant invention also encompasses the use of a therapeutically or prophylactically effective amount, as appropriate, of an HMG-CoA reductase inhibitor for the preparation of a medicament for combined use with a therapeutically or prophylactically effective amount, as appropriate, of an ACAT inhibitor. for preventing or reducing the risk for onset of Alzheimer's disease, treating or slowing the progression of Alzheimer's disease, preventing or reducing Aβ formation, as well as for reducing a patient's Aβ level.

The instant invention also encompasses the use of an effective amount of an HMG-CoA reductase inhibitor and an effective amount of an ACAT inhibitor for the preparation of a medicament useful for preventing or reducing the risk for onset of Alzheimer's disease, treating or slowing the progression of Alzheimer's disease, preventing or reducing Aβ formation, or for reducing a patient's Aβ level. The effective amount of each active agent is either a therapeutically or prophylactically effective amount, as appropriate, depending on whether the medicament can be used in methods of treatment or prevention.

The instant invention also encompasses the use of a therapeutically or prophylactically effective amount, as appropriate, of an HMG-CoA reductase inhibitor for the preparation of a medicament for combined use with a therapeutically or prophylactically effective amount, as appropriate, of an ACAT inhibitor, wherein the medicament is useful for treatment or prevention of any of the conditions described herein. The instant invention further encompasses the use of a therapeutically or prophylactically effective amount, as appropriate, of an ACAT inhibitor for the preparation of a medicament for combined use with a therapeutically or prophylactically effective amount, as appropriate, of an HMG-CoA reductase inhibitor, wherein the medicament is useful for treatment or prevention of any of the conditions described herein.

The medicament or pharmaceutical drug combination comprised of the an HMG-CoA reductase inhibitor and the ACAT inhibitor may also be prepared with one or more additional active agents, such as those described above.

While the invention has been described and illustrated with reference to certain particular embodiments thereof, those skilled in the art will appreciate that various changes, modifications and substitutions can be made therein without departing from the spirit and scope of the invention. For example, specific effective dosage amounts other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method for preventing Aβ formation comprising administering a prophylactically effective amount of an HMG-CoA reductase inhibitor in combination with a prophylactically effective amount of an ACAT inhibitor to patient in need thereof.

2. A method for reducing Aβ formation comprising administering a therapeutically effective amount of an HMG-CoA reductase inhibitor in combination with a therapeutically effective amount of an ACAT inhibitor to patient in need thereof.

3. A method for preventing or reducing the risk for onset of Alzheimer's disease comprising administering a prophylactically effective amount of an HMG-CoA reductase inhibitor in combination with a prophylactically effective amount of an ACAT inhibitor to patient in need thereof

4. A method for treating Alzheimer's disease comprising administering a therapeutically effective amount of an HMG-CoA reductase inhibitor in combination with a therapeutically effective amount of an ACAT inhibitor to patient in need thereof.

5. The method according to claim 4 wherein the HMG-CoA reductase inhibitor is selected from the lactone and dihydroxy open-acid forms of lovastatin, simvastatin, pravastatin, fluvastatin, atorvastatin, rosuvastatin, pitavastatin and the pharmaceutically acceptable salts and esters thereof.

6. The method of claim 5 wherein the HMG-CoA reductase inhibitor is selected from the lactone and dihydroxy open-acid forms of simvastatin and the pharmaceutically acceptable salts and esters thereof.

7. The method of claim 5 wherein the HMG-CoA reductase inhibitor is selected from the lactone and dihydroxy open-acid forms of lovastatin and the pharmaceutically acceptable salts and esters thereof.

8. The method of claim 4 wherein the ACAT inhibitor is selected from the group consisting of: 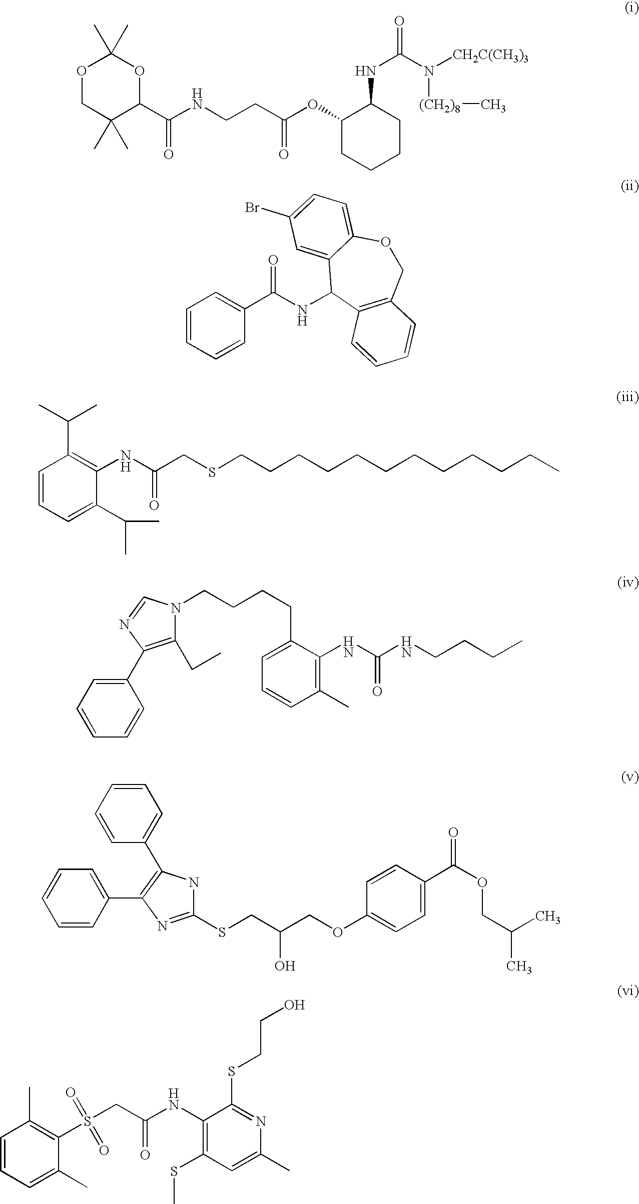 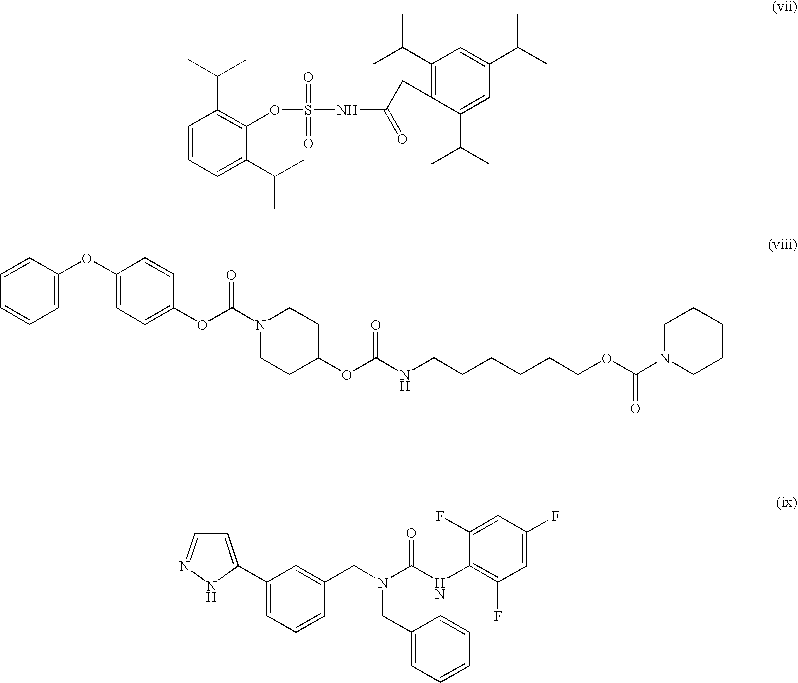
and the pharmaceutically acceptable salts and esters thereof.
